(19) United States
(12) Patent Application Publication
(10) Pub. No.: US 2007/0249925 A1
(43) Pub. Date: Oct. 25, 2007

(54) X-Ray Diagnostic Device for Mammography

(76) Inventors: Martin Hoheisel, Erlangen (DE); Thomas Mertelmeier, Erlangen (DE); Marcus Pfister, Bubenreuth (DE)

Correspondence Address: BRINKS HOFER GILSON & LIONE, P.O. BOX 10395, CHICAGO, IL, 60610, US

(21) Appl. No.: 11/661,889

(22) PCT Filed: Aug. 29, 2005
(86) PCT No.: PCT/EP05/54234

§ 371 Date: Mar. 1, 2007

(51) Int. Cl. A61B 5/05

(52) U.S. Cl. 600/407

(57) ABSTRACT

An X-ray diagnostic device is provided. The X-ray diagnostic device includes a control device that is operable to optimally adjust a radiation of an X-ray emitter for a particular patient, and a measuring device. The measuring device is coupled to the control device. The measuring device is operable to determine a tissue composition of a body part to be examined. The control device is operable to optimally adjust the radiation of the X-ray emitter for the particular patient on the basis of the measured tissue composition.

The present patent document is a §371 nationalization of PCT Application Serial Number PCT/EP2005/054234, filed Aug. 29, 2005, designating the United States, which is hereby incorporated by reference. This patent document also claims the benefit of DE10 2004 043 032.2, filed Sep. 6, 2004, which is also hereby incorporated by reference.

### BACKGROUND

The present embodiments relate to an X-ray diagnostic device.

In mammography, the difficulty in determining the optimal exposure is that the female breast is a highly variable organ. Breast size, or in the compressed case the breasts thickness, is highly variable. The breast's composition also ranges from very high-fat tissue to glandular tissue. Precise adaptation of the exposure parameters is necessary because of the breast being a highly variable organ.

Film-foil systems were used in conventional mammography. Recently, digital systems with solid-state detectors have become increasingly common.

In film-based systems, precise exposure to light is necessary. A slight overexposure or underexposure leads to pronounced losses in contrast recognition of details of interest. A measurement cell is therefore placed in the beam path downstream of the film cassette. The measurement cell measures the radiation not absorbed by the amplifier foil and uses the exposure time for control. However, the beam hardening affects the measured values in such a way that incorrect exposures can occur, depending on the thickness of the breast. This problem can be solved with the aid of a double detector.

Digital solid-state detectors are linear over wide dosage ranges and are much more tolerant to variable exposure. With a high dose, overmodulation plays an interfering role as a result. At a low dose, electronic noise plays an interfering role. One concept for controlling mammography with a detector of this kind has been described in German Patent Disclosure DE 100 19 242 A1.

When adjusting the radiation quality or the high voltage of the X-ray tube in either the film-foil system or the system with a solid-state detector, only the thickness of the compressed breast is definitive. The set voltage is therefore not always optimal.

U.S. Pat. No. 6,157,697 discloses a device with which both X-ray images and 3D distributions of the electrical impedance can be recorded. The measuring arrangement picks up a three-dimensional distribution of impedance values. A control unit correctly triggers the many electrodes present in accordance with a defined pattern or for selection of sets of parameters, stored in memory in the control unit, for the aforementioned operating parameters using a keyboard. Alternatively, the operating parameters of the X-ray emitter are directly input via the keyboard.

### SUMMARY

The present embodiments may obviate one or more of the drawbacks or limitations inherent in the related art. For example, in one embodiment, variable consistency and composition of the body part to be X-rayed is detected and used for optimally adjusting the radiation of an X-ray emitter.

An X-ray diagnostic device includes a measuring arrangement, coupled to the control device, for determining the tissue composition of the body part to be examined. The measuring arrangement includes electrodes for placement on the body part to be examined and is coupled to the control device. In one embodiment, the measuring arrangement includes a body fat analyzer. The control device is embodied for optimally adjusting the radiation of the X-ray emitter for the particular patient on the basis of the measured tissue composition.

Optimized X-ray examination can be performed once the system has been suitably pre-calibrated because the tissue composition and the proportion of fat in the breast to be examined are taken into account. For each combination of breast thickness and proportion of fat, optimal values for the anode and filter material, the filter thickness, and the tube voltage are determined by simulation or phantom measurement and placed in tables. In one embodiment, the optimal values are stored in memory in the control device.

The body fat analyzer can be an electrical impedance measuring device. The examination region can determine the skin resistance and the tissue resistance of the examination region via two respective pairs of impedance electrodes.

The electrodes can be placed on diametrically opposite sides of the compressed breast in order to perform the body fat analysis. In another embodiment, the electrodes and their electric leads may include X-ray-permeable material and be integrated into the compression plates.

For example, the electrodes and the electric leads may comprise aluminum, an aluminum-magnesium alloy, or an organic conductive polymer, such as polyaniline or PEDOT (polyethylenethioxythiophene). When the electrodes are integrated into the compression plates the electrodes can be used to position the breast correctly (incorrect positioning is one of the most frequent reasons for having to repeat the imaging procedure).

The two electrodes located on the same side of the breast can measure individual skin resistance. The tissue resistance is measured with the respective diametrically opposed electrodes. The two measurements are used to determine the tissue composition of the breast, in particular the fat content. A statement can be made about the proportions of glandular tissue and fatty tissue. The statement can be used, together with the breast thickness and other known variables, to determine the optimal X-ray parameters for the particular patient.

Additional patient-specific data can be processed in the control device as parameters for optimizing the emitter setting, such as the thickness of the body part, that is, the thickness of the breast compressed between the compression plates; the compressive force; the hormonal and therapy status; the age of the patient; and/or the presence of implants.

### BRIEF DESCRIPTION OF THE FIGURES

FIG. 1 is a schematic view of one embodiment of an X-ray diagnostic device for mammography;

FIG. 2 is a fragmentary view of one embodiment of the compression plates and the electrodes serving to ascertain the tissue composition, the electrodes being separate from the compression plates; and

FIG. 3 is a fragmentary view of one embodiment of the compression plates and the electrodes serving to ascertain the tissue composition, the electrodes being integrated with compression plates.

### DETAILED DESCRIPTION

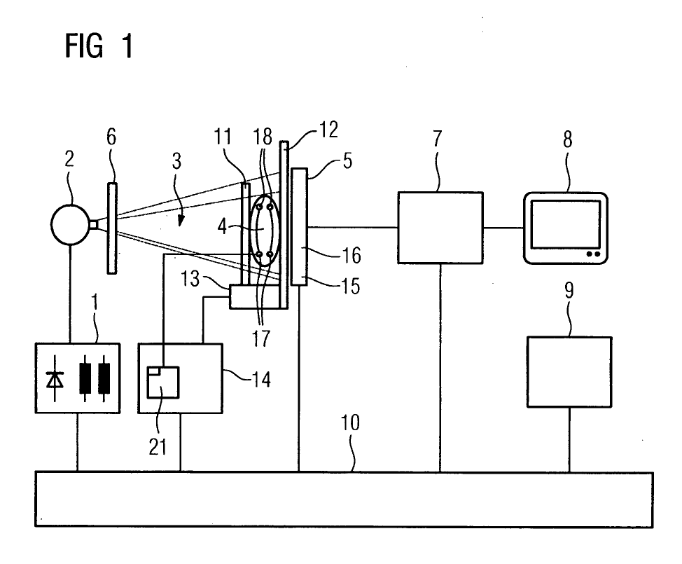

As shown in FIG. 1, an X-ray mammography system includes an X-ray tube 2 supplied with high voltage and heating voltage by a high-voltage generator 1. The X-ray tube 2 generates a conical X-ray beam 3, which penetrates a patient's breast 4 to be examined. The X-ray beam 3 generates radiation images on a digital solid-state image converter 5 that is sensitive to X-radiation 3. The solid-state image converter 5 includes, for example, switch elements of amorphous silicon (a-Si:H) and has pixels arranged in a matrix.

Interchangeable filters 6 are disposed near the X-ray tube 2 and in the X-ray beam 3. The output signal of the solid-state image converter 5 is delivered to an image processing system 7. The image processing system 7 may have converters, image memories, and processing circuits. The image processing system 7 is connected to a monitor 8 for reproduction of the X-ray images detected. The user surface 9 communicates with the other components of the X-ray diagnostic device via a system control and communication unit 10.

The breast 4 to be examined is pressed by a compression plate 11 against a cover plate 12 on the inlet side of the solid-state image converter 5. A sensor 13 measures the thickness of the compressed breast 4. The measured thickness is forwarded to a control device 14. The control device 14 may also be part of the high-voltage generator 1 or the image processing system 7. Part of the X-ray beam 3 penetrates the breast 4 and, attenuated, strikes a region 16 of the solid-state image converter 5. Laterally (adjacent) of the breast 4, part of the X-ray beam 3 strikes a region 15 of the solid-state image converter 5 unattenuated or directly.

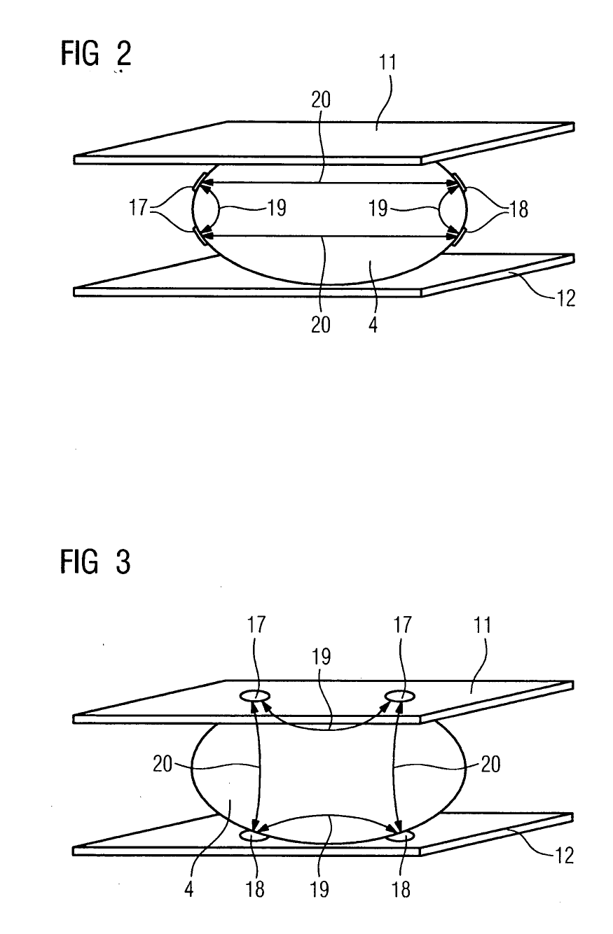

As shown in FIG. 1, the X-ray diagnostic device includes two pairs 17 and 18 of electrodes and each pair is to be disposed on one of the diametrically opposed sides of the breast compressed between the compression plates 11 and 12. The measurement of the skin resistance is done via the respective electrodes 17, 17 and 18, 18 disposed on the same side of the breast The fat content is measured via a respective one of the electrodes 17 and 18 with a current path extending transversely through the breast. As shown in FIGS. 1 and 2, the electrodes 17, 17 and 18, 18 are separate components and are arranged on diametrically opposed free sides of the breast.

In one embodiment, as shown in FIG. 3, the electrodes 17 and 18 are integrated into the compression plates 11 and 12. The electrodes 17, 18 and their leads comprise X-ray-permeable material. The arrows 19 and 20 show the type of wiring 19 of the electrodes for measuring the skin resistance and the wiring 20 for measuring the fat content.

In the control device 14, the optimal values for the anode material, filter 6, tube voltage, tube current, and the duration of the pulse of X-radiation, values that pertain to the thickness and the known geometry, that is, the spacing between the X-ray focus and the solid-state image converter, are taken from a table that includes a table memory 20. For every combination of breast thickness and proportion of fat, the optimal value for the anode and filter material, the filter thickness, and the tube voltage have been determined in advance by simulation of phantom measurement and determined in table form and stored in the table memory 21, so that the measurement values, which are understood also to be processed by electronics, of the electrodes 17 and 18 are automatically taken into account in optimizing the emitter setting.

The present embodiments are not limited to the exemplary embodiments shown. For example, it does not matter whether the breast is compressed between vertically placed compression plates or between horizontal compression plates. The tissue composition of the body part examined may be used to adjust the X-ray emitter when a film-foil system is used instead of a solid-state image converter.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

1. An X-ray diagnostic device comprising:
a control device operable to optimally adjust a radiation of a X-ray emitter for a particular patient; and
a measuring device, the measuring device being coupled to the control device,
wherein the measuring device is operable to determine a tissue composition of a body part to be examined, the control device being operable to optimally adjust the radiation of the X-ray emitter for the particular patient on the basis of the measured tissue composition.

2. The X-ray diagnostic device as defined by claim 1, wherein the measuring device comprises a body fat analyzer that includes electrodes for placement on the body part to be examined and are coupled to the control device.

3. The X-ray diagnostic device as defined by claim 2, wherein the body fat analyzer is an electrical impedance measuring device that includes two respective pairs of impedance electrodes, the electrical impedance measuring device being operable to determine the skin resistance and the tissue resistance of the examination region are determined.

4. The X-ray diagnostic device as defined by claim 2, wherein the electrodes and respective electric leads are X-ray-permeable material and are integrated into the compression plates.

5. The X-ray diagnostic device as defined by claim 1, wherein the control device is operable to process additional patient-specific data as parameters for optimizing the emitter setting.

6. The X-ray diagnostic device as defined by claim 1, wherein the control device includes a memory that is operable to store tables of optimal values for an anode and filter material, a filter thickness, and tube voltage determined by simulation or phantom measurement of each combination of breast thickness and proportion of fat.

7. The X-ray diagnostic device as defined by claim 3, wherein the impedance electrodes and respective electric leads that include X-ray-permeable material are integrated into the compression plates.

8. The X-ray diagnostic device as defined by claim 5, wherein the additional patient-specific data includes a thickness of the body part; the compressive force; the hormonal and therapy status; the age of the patient; and/or the presence of implants.

9. The X-ray diagnostic device as defined by claim 8, wherein the thickness of the body part is a thickness of a breast compressed between the compression plates.

10. The X-ray diagnostic device as defined by claim 4, wherein the control device is operable to process additional patient-specific data as parameters for optimizing the emitter setting.

11. The X-ray diagnostic device as defined by claim 10, wherein the additional patient-specific data includes a thickness of the body part, a compressive force, a hormonal and therapy status, an age of the patient, a presence of implants, or combinations thereof.

12. The X-ray diagnostic device as defined by claim 11, wherein the thickness of the body part is a thickness of a breast compressed between the compression plates.

13. The X-ray diagnostic device as defined by claim 7, wherein the control device is operable to process additional patient-specific data as parameters for optimizing the emitter setting.

14. The X-ray diagnostic device as defined by claim 13, wherein the additional patient-specific data includes a thickness of the body part, a compressive force, a hormonal and therapy status, an age of the patient, a presence of implants, or combinations thereof.

15. The X-ray diagnostic device as defined by claim 14, wherein the thickness of the body part is a thickness of a breast compressed between the compression plates.

16. The X-ray diagnostic device as defined by claim 12, wherein the control device includes a memory that is operable to store tables of optimal values for an anode and filter material, a filter thickness, and a tube voltage determined by simulation or phantom measurement of each combination of breast thickness and proportion of fat.

17. The X-ray diagnostic device as defined by claim 15, wherein the control device includes a memory that is operable to store tables of optimal values for an anode and filter material, a filter thickness, and a tube voltage determined by simulation or phantom measurement of each combination of breast thickness and proportion of fat.
